(19) United States
(12) Patent Application Publication
(10) Pub. No.: US 2017/0151039 A1
(43) Pub. Date: Jun. 1, 2017

(54) METHOD OF MANUFACTURING SCAFFOLD FOR TREATMENT OF TOOTH EXTRACTION SOCKET

(71) Applicant: THE CATHOLIC UNIVERSITY OF KOREA INDUSTRY-ACADEMIC COOPERATION FOUNDATION, Seoul (KR)

(72) Inventors: Sang Hwa LEE, Seoul (KR); Jin Hyung SHIM, Incheon (KR)

(21) Appl. No.: 15/123,120

(22) PCT Filed: Jul. 7, 2016
(86) PCT No.: PCT/KR2016/007347

§ 371 Date: Sep. 1, 2016

(30) Foreign Application Priority Data
Jul. 8, 2015 (KR) 10-2015-0097345

(51) Int. Cl. A61C 8/02; A61B 6/03; A61B 6/14; A61C 13/00

(52) U.S. Cl. CPC A61C 8/0006; A61C 13/0004; A61C 13/0019; A61B 6/032; A61B 6/14

(57) ABSTRACT

Provided is a technique of accurately forming a shape of a scaffold demanding high precision and fast manufacturing the scaffold with low costs, so that the scaffold for dental treatment may be immediately placed after tooth extraction and may be accurately placed in a tooth extraction socket. A method of manufacturing a scaffold for treatment of a tooth extraction socket includes manufacturing, by using a three-dimensional (3D) printer, a 3D model comprising alveolar bones and teeth, which are distinguished therebetween, from a medical image file that is a medical image file (DICOM file) of dental computerized tomography (CT) data; performing virtual tooth-extraction by removing, from the manufactured 3D model, a region corresponding to a tooth in a tooth-extraction target area; and manufacturing, by using the 3D printer, a scaffold to be placed in an actual tooth extraction socket according to a shape of a tooth extraction socket that exists in the manufactured 3D model as a result of the virtual tooth-extraction.

### TECHNICAL FIELD

The present disclosure relates to a technique of manufacturing a scaffold to be inserted into a tooth extraction socket after tooth-extraction to help treatment, and more particularly, to a technique of fast and accurately manufacturing a scaffold to be immediately placed after tooth-extraction in a tooth extraction process, and to be stably customized into a tooth-extraction target area of a patient.

### BACKGROUND ART

Although a tooth extraction socket formed after tooth extraction is spontaneously cured, the tooth extraction socket causes a loss of a height and width of alveolar bone during a curing process. The loss highly influences following implantation of a dental implant and a prognosis thereof. Accordingly, various bone grafts and barrier membranes using techniques have been used to maintain the form of alveolar bone. In particular, the techniques have been used to preserve the form of alveolar bone by immediately implanting a dental implant and grafting bones as soon as a tooth is extracted in the anterior teeth area, and to reduce a treatment period.

However, when there is a loss of a veneer block bone due to lesions, external injuries, etc., it is difficult to fix bone grafts and a dental implant and a treatment period is prolonged.

In this regard, Korean Patent Publication No 10-1527934 discloses a technique of manufacturing a scaffold for dental treatment by using various materials.

The conventional techniques disclose technical features of a dental implant or a scaffold for dental treatment, wherein the dental implant or the scaffold have osseointegration and osteogenesis effects and are capable of releasing an osteogenesis stimulating material.

However, the conventional techniques only describe specific materials of a scaffold. Accordingly, there is no research on a technique of allowing a scaffold to be accurately placed in a tooth extraction socket of a patient.

### DETAILED DESCRIPTION OF THE INVENTION

### Technical Problem

The present disclosure provides a technique of accurately forming a shape of a scaffold demanding high precision and fast manufacturing the scaffold with low costs, so that the scaffold for dental treatment may be immediately placed after tooth-extraction and may be accurately placed in a tooth extraction socket.

Technical Solution

According to an embodiment of the present disclosure, there is provided a method of manufacturing a scaffold for treatment of a tooth extraction socket, the method including manufacturing, by using a three-dimensional (3D) printer, a 3D model including alveolar bones and teeth, which are distinguished therebetween, from a medical image file that is a medical image file (DICOM file) of dental computerized tomography (CT) data; performing virtual tooth-extraction by removing, from the manufactured 3D model, a region corresponding to a tooth in a tooth-extraction target area; and manufacturing, by using the 3D printer, a scaffold to be placed in an actual tooth extraction socket according to a shape of a tooth extraction socket that exists in the manufactured 3D model as a result of the virtual tooth-extraction.

According to another embodiment of the present disclosure, there is provided a method of manufacturing a scaffold for treatment of a tooth extraction socket, the method including receiving, from a computerized tomography (CT) imaging apparatus, a medical image file (DICOM file) of dental computerized tomography (CT) data; generating image data of a scaffold to be placed in a tooth extraction socket formed when a tooth in a tooth-extraction target area is extracted, by using a region corresponding to the tooth in the tooth-extraction target area in the medical image file; and manufacturing the scaffold corresponding to the generated image data of the scaffold, by using a three-dimensional (3D) printer.

### Advantageous Effects of the Invention

According to embodiments of the present disclosure, a shape of a tooth extraction socket before tooth-extraction may be accurately predicted by using a medical image file included in computerized tomography (CT) imaged teeth data obtained for dental treatment before the tooth-extraction, so that a scaffold fixable into the tooth extraction socket may be manufactured.

Therefore, the scaffold may be manufactured to be immediately placed as soon as an actual tooth of a patient is extracted, thereby enabling fast and accurate treatment. In particular, since the scaffold is manufactured to be placed to accurately correspond to a shape of a tooth extraction socket which may vary according to extraction when the tooth of the patient is extracted, an effect of treatment may be significantly increased.

### DESCRIPTION OF THE DRAWINGS

FIG. 1 is a flowchart of a method of manufacturing a scaffold for treatment of a tooth extraction socket, according to an embodiment.

FIG. 2 is a flowchart of a method of manufacturing a scaffold for treatment of a tooth extraction socket, according to another embodiment.

FIG. 3 illustrates a test example of manufacturing a scaffold for treatment of a tooth extraction socket according to the embodiments.

### BEST MODE

Hereinafter, a method of manufacturing a scaffold for treatment of a tooth extraction socket according to embodiments of the present disclosure will now be described with reference to accompanying drawings.

For the purposes of promoting an understanding of the principles of the invention, well-known functions or constructions are not described in detail. It should be understood that embodiments described herein should be considered in a descriptive sense only and not for purposes of limitation. Descriptions of features or aspects within each embodiment should typically be considered as available for other similar features or aspects in other embodiments.

Throughout the specification, like reference numerals refer to like elements throughout and redundant descriptions thereof are not provided here.

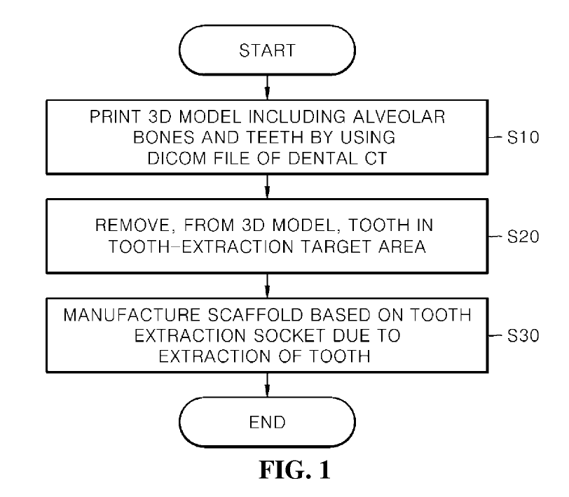

FIG. 1 is a flowchart of a method of manufacturing a scaffold for treatment of a tooth extraction socket, according to an embodiment.

Referring to FIG. 1, the method of manufacturing a scaffold for treatment of a tooth extraction socket according to the present embodiment includes manufacturing, by using a three-dimensional (3D) printer, a 3D model including alveolar bones and teeth, which are distinguished therebetween, based on a medical image file (a digital imaging and communications in medicine (DICOM) file) of dental CT data (operation S10).

Dental CT is also referred to as cone beam computed tomography (CBCT) that is a scanner capable of scanning inside a tooth to be three-dimensionally recognizable. When a dental CT is performed by using a CT imaging apparatus, dental CT data is generated, and the data includes a medical image file (DICOM file) corresponding to a 3D image of the tooth.

A terminal of a manager receives the medical image file from the CT imaging apparatus, and a 3D printer connected to the terminal of the manager outputs and manufactures a 3D model corresponding to the medical image file.

In a medical image file that is generated as a CT imaging result, basically, clear white parts represent teeth, and less clear white parts such as soft white represent alveolar bones. Therefore, the alveolar bones may be distinguished from the teeth in the dental CT data of a patient, by using the medical image file.

When the 3D printer outputs a 3D model by using the medical image file, the 3D printer may output a 3D model where the alveolar bones are distinguished from the teeth to the extent that extraction of the teeth is available, so that the 3D printer may manufacture the 3D model according to the present embodiment. That is, in all embodiments of the present disclosure, a 3D model indicates a virtual object of a shape of an alveolar bone and tooth of a patient manufactured by using a 3D printer based on a medical image file.

When the operation S10 is performed and thus the 3D model is manufactured thereafter, operation S20 is performed in which virtual tooth-extraction is performed by removing, from the 3D model, a region corresponding to a tooth in a tooth-extraction target area of a patient.

To perform the virtual tooth-extraction in the 3D model means that the tooth is extracted not from an actual patient but from the object manufactured as the 3D model. The medical team determines a position of tooth-extraction based on the dental CT data, and then the operation S20 is performed by performing the tooth-extraction in the 3D model.

When the operation S20 is performed, the object in the 3D model is configured to have the extraction-target tooth extracted. As a result, a tooth extraction socket due to the extraction is formed in the 3D model. Afterward, operation S30 is performed in which a scaffold to be placed in an actual tooth extraction socket is manufactured by using the 3D printer, according to a shape of the tooth extraction socket that exists in the 3D model as the result of the virtual tooth-extraction.

In more detail, in the operation S30, a 3D image of the scaffold to be manufactured may be generated according to the shape of the tooth extraction socket existing in the 3D model, the generated image may be transmitted and requested for an output to the 3D printer, and then the scaffold may be manufactured by using the 3D printer.

For example, since the scaffold is manufactured by using a strip for a 3D printing output, the strip including bio-affinitive polymer (e.g., polycaprolactone (PCL)), it is possible to manufacture the bio-affinitive scaffold and to increase an effect on treatment.

According to the flowchart, the virtual tooth-extraction may be performed on the 3D model that is copied and manufactured to match with the shape of the alveolar bone and tooth of the patient, and the scaffold may be manufactured by using the 3D printer so that the scaffold may be accurately implanted to the shape of the tooth extraction socket formed due to the tooth-extraction.

After the scaffold is manufactured via the aforementioned procedure, additional operation of placing the scaffold in an actual tooth extraction socket of the patient may be performed. In this regard, when an alveolar bone of a scaffold placement target part is heavily absorbed, the scaffold may not be appropriately fixed.

In this case, the scaffold may be securely fixed to the alveolar bone by using a member such as a screw, or the like. The screw may be directly fixed to the scaffold placed at a grafting part or may be manufactured during the manufacture of the scaffold and then may be placed.

Accordingly, without extracting an actual tooth of a patient, not a scaffold having a fixed shape but the scaffold that is accurately fixable into a tooth extraction socket formed after extraction of the actual tooth of the patient may be manufactured regardless of the tooth extraction. Accordingly, as soon as the actual tooth of the patient is extracted, the scaffold may be immediately placed. In addition, since the scaffold can be accurately manufactured to be exactly placed in the actual tooth extraction socket of the patient, an effect of treatment may be maximized due to stable placement into the tooth extraction socket.

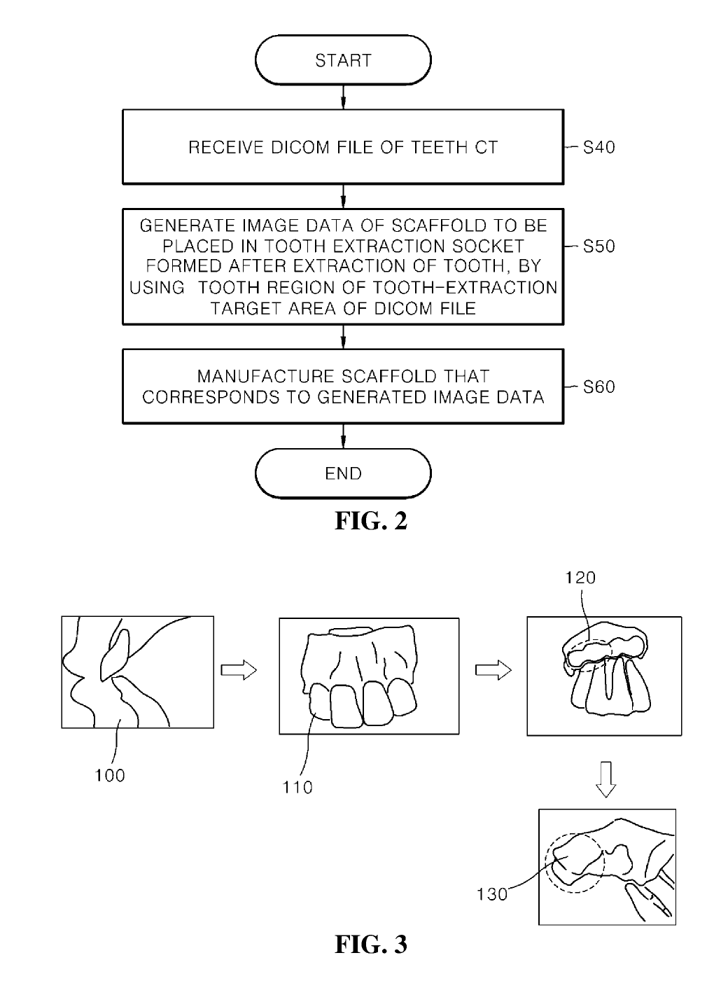

FIG. 2 is a flowchart of a method of manufacturing a scaffold for treatment of a tooth extraction socket, according to another embodiment. Hereinafter, detailed descriptions, which are the same as the aforementioned contents of FIG. 1, will be omitted.

Referring to FIG. 2, the method of manufacturing a scaffold for treatment of a tooth extraction socket according to the present embodiment includes receiving a medical image file of dental CT data from a CT imaging apparatus (operation S40).

When the operation S10 is performed, unlike to the embodiment of FIG. 1, operation S50 is performed in which image data of a scaffold that is fixable into a tooth extraction socket formed after extraction of a tooth in a tooth-extraction target area may be generated by using an image of a region of the received medical image file, wherein the region corresponds to the tooth in the tooth-extraction target area.

That is, referring to FIG. 1, the 3D model corresponding to the medical image file is generated and then the image of the scaffold is generated therefrom, whereas in the present embodiment of FIG. 2, an image of the tooth extraction socket formed after the tooth extraction is predicted from the medical image file, and the image data of the scaffold is generated therefrom.

In more detail, as described above, the image of the medical image file is configured in such a manner that the alveolar bones and the teeth are distinguished therebetween. In this regard, when the image corresponding to the region in the extraction-target tooth is removed, a shape of the tooth extraction socket to be formed thereof may be predicted, accordingly, the image data may be generated from the medical image file, wherein the image data includes data about a detailed shape of the scaffold that is fixable into the tooth extraction socket formed as a result of the tooth extraction.

After the operation S50 is performed, operation S60 is performed in which the generated image data of the scaffold is transmitted to the 3D printer and thus the scaffold corresponding to the generated image data of the scaffold is manufactured by using the 3D printer.

According to the present embodiment, similar to the previous embodiment, the virtual tooth-extraction may be performed on the 3D model that is duplicated to match the shape of the alveolar bone and tooth of the patient, and the scaffold may be manufactured by using the 3D printer so that the scaffold may be accurately implanted to match the shape of the tooth extraction socket formed due to the tooth-extraction.

Accordingly, without extracting an actual tooth of a patient, not a scaffold having a fixed shape but the scaffold that is accurately fixable into a tooth extraction socket formed after extraction of the actual tooth of the patient may be manufactured regardless of the tooth extraction. Accordingly, as soon as the actual tooth of the patient is extracted, the scaffold may be immediately placed. In addition, since the scaffold can be accurately manufactured to be exactly placed in the actual tooth extraction socket of the patient, an effect of treatment may be maximized due to stable placement into the tooth extraction socket.

FIG. 3 illustrates a test example of manufacturing a scaffold for treatment of a tooth extraction socket according to the embodiment of FIG. 1.

Referring to FIG. 3, an image 100 of a CT imaged teeth may be generated as a result of CT imaging. In this regard, the medical image file is received and then is transmitted to the 3D printer, so that the 3D model 110 is manufactured.

Afterward, by performing the aforementioned features of the embodiment of FIG. 1, it is possible to virtually reproduce a tooth extraction socket formed by performing tooth extraction on the 3D model 110. At this time, an image of a scaffold that is fixable into the tooth extraction socket may be generated, and a scaffold 130 may be manufactured based on the image, by using the 3D printer.

As illustrated in FIG. 3, shapes of alveolar bones and teeth of a patient are exactly reproduced as a 3D model and a scaffold is manufactured based on the 3D model, so that the scaffold having high accuracy with respect to placement may be manufactured without directly extracting or measuring a tooth of the patient.

It will be apparent that all elements of the one or more embodiments of the present disclosure are not limited to be combined or to operate as one combination. That is, all elements may be selectively combined and may operate as one within the scope of the present disclosure.

In addition, when a part “includes”, “comprises”, “is configured of”, or “has” an element, unless there is a particular description contrary thereto, the part can further include other elements, not excluding the other elements. Unless expressly described otherwise, all terms including descriptive or technical terms which are used herein should be construed as having meanings that are obvious to one of ordinary skill in the art. Also, terms that are defined in a general dictionary and that are used in the following description should be construed as having meanings that are equivalent to meanings used in the related description, and unless expressly described otherwise herein, the terms should not be construed as being ideal or excessively formal.

While the present disclosure has been particularly shown and described with reference to exemplary embodiments thereof, it will be understood by one of ordinary skill in the art that various changes in form and details may be made therein without departing from the spirit and scope of the following claims. Therefore, the scope of the present disclosure is defined not by the detailed description of the present disclosure but by the appended claims, and all differences within the scope will be construed as being included in the present disclosure.

1. A method of manufacturing a scaffold for treatment of a tooth extraction socket, the method comprising:
manufacturing, by using a three-dimensional (3D) printer, a 3D model comprising alveolar bones and teeth, which are distinguished therebetween, from a medical image file that is a medical image file (DICOM file) of dental computerized tomography (CT) data;
performing virtual tooth-extraction by removing, from the manufactured 3D model, a region corresponding to a tooth in a tooth-extraction target area; and
manufacturing, by using the 3D printer, a scaffold to be placed in an actual tooth extraction socket according to a shape of a tooth extraction socket that exists in the manufactured 3D model as a result of the virtual tooth-extraction.

2. The method of claim 1, wherein the manufacturing of the scaffold by using the 3D printer comprises manufacturing the scaffold by using a strip for a 3D printing output, the strip comprising bio-affinitive polymer such as polycaprolactone (PCL).

3. A method of manufacturing a scaffold for treatment of a tooth extraction socket, the method comprising:
receiving, from a computerized tomography (CT) imaging apparatus, a medical image file (DICOM file) of dental computerized tomography (CT) data;
generating image data of a scaffold to be placed in a tooth extraction socket formed when a tooth in a tooth-extraction target area is extracted, by using a region corresponding to the tooth in the tooth-extraction target area in the medical image file; and
manufacturing the scaffold corresponding to the generated image data of the scaffold, by using a three-dimensional (3D) printer.

4. The method of claim 3, wherein the manufacturing of the scaffold comprises manufacturing the scaffold by using a strip for a 3D printing output, the strip comprising bio-affinitive polymer such as polycaprolactone (PCL).
